(19) United States
(12) Patent Application Publication
(10) Pub. No.: US 2008/0260651 A1
(43) Pub. Date: Oct. 23, 2008

(54) CONTRAST AGENTS

(75) Inventors: Veronique Morisson-Iveson, Buckinghamshire (GB); Joanna Marie Passmore, Buckinghamshire (GB)

Correspondence Address: GE HEALTHCARE, INC., IP DEPARTMENT, 101 CARNEGIE CENTER, PRINCETON, NJ, 08540-6231, US

(73) Assignee: GE HEALTHCARE AS, Oslo (NO)

(21) Appl. No.: 12/103,032

(22) Filed: Apr. 15, 2008

(30) Foreign Application Priority Data
Apr. 17, 2007 (NO) 20071949

(51) Int. Cl. A61K 49/04; C07D 207/12; C07D 211/40

(52) U.S. Cl. 424/9.44; 548/539; 546/220; 424/9.1

(57) ABSTRACT

The present invention relates to a class of compounds and to diagnostic compositions containing such compounds where the compounds are iodine containing compounds. More specifically the iodine containing compounds are chemical compounds containing an aliphatic N-heterocyclic central moiety such as pyrrolidine or piperidine heterocycles allowing for the arrangement of three iodinated phenyl groups bound thereto.

The invention also relates to the use of such diagnostic compositions as contrast agents in diagnostic imaging, in particular in X-ray imaging, and to contrast media containing such compounds.

### FIELD OF THE INVENTION

The present invention relates to a class of compounds and to diagnostic compositions containing such compounds where the compounds are iodine containing compounds. More specifically the iodine containing compounds are chemical compounds containing an aliphatic N-heterocyclic central moiety such as pyrrolidine or piperidine heterocycles allowing for the arrangement of three iodinated phenyl groups bound thereto.

The invention also relates to the use of such diagnostic compositions as contrast agents in diagnostic imaging and in particular in X-ray imaging and to contrast media containing such compounds.

### BACKGROUND OF THE INVENTION

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials formulated as contrast media into the body region being imaged.

Thus in X-ray early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade name Gastrografen™), ionic dimers such as ioxaglate (marketed e.g. under the trade name Hexabrix™), nonionic monomers such as iohexyl (marketed e.g. under the trade name Omnipaque™), iopamidol (marketed e.g. under the trade name Isovue™), iomeprol (marketed e.g. under the trade name Iomeron™) and the non-ionic dimer iodixanol (marketed under the trade name and Visipaque™).

The most widely used commercial non-ionic X-ray contrast agents such as those mentioned above are considered safe. Contrast media containing iodinated contrast agents are used in more that 20 millions of X-ray examinations annually in the USA and the number of adverse reactions is considered acceptable. However, since a contrast enhanced X-ray examination will require up to about 200 ml contrast media administered in a total dose, there is a continuous drive to provide improved contrast media.

The utility of the contrast media is governed largely by its toxicity, by its diagnostic efficacy, by adverse effects it may have on the subject to which the contrast medium is administered, and by the ease of storage and ease of administration. Since such media are conventionally used for diagnostic purposes rather than to achieve direct therapeutic effect, it is generally desirable to provide media having as little as possible effect on the various biological mechanisms of the cells or the body as this will lead to lower toxicity and lower adverse clinical effect. The toxicity and adverse biological effects of a contrast medium are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the contrast agent, the osmolality of the contrast medium and the ionic composition or lack thereof of the contrast medium.

Desirable characteristics of an iodinated contrast agent are low toxicity of the compound itself (chemotoxicity), low viscosity of the contrast medium wherein the compound is dissolved, low osmolality of the contrast medium and a high iodine content (frequently measured in g iodine per ml of the formulated contrast medium for administration). The iodinated contrast agent must also be completely soluble in the formulation medium, usually an aqueous medium, and remain in solution during storage.

The osmolalities of the commercial products, and in particular of the non-ionic compounds is acceptable for most media containing dimers and non-ionic monomers although there is still room for improvement. In coronary angiography for example, injection into the circulatory system of a bolus dose of contrast medium has caused severe side effects. In this procedure contrast medium rather than blood flows through the system for a short period of time, and differences in the chemical and physiochemical nature of the contrast medium and the blood that it replaces can cause undesirable adverse effects such as arrhythmias, QT prolongation and reduction in cardiac contractive force. Such effects are seen in particular with ionic contrast agents where osmotoxic effects are associated with hypertonicity of the injected contrast medium. Contrast media that are isotonic or slightly hypotonic with the body fluids are particularly desired. Low osmolar contrast media have low renal toxicity which is particularly desirable. The osmolality is a function of the number of particles per volume unit of the formulated contrast medium.

To keep the injection volume of the contrast media as low as possible it is highly desirable to formulate contrast media with high concentration of iodine/ml, and still maintain the osmolality of the media at a low level, preferably below or close to isotonicity. The development of non-ionic monomeric contrast agents and in particular non-ionic bis(triiodophenyl) dimers such as iodixanol (EP patent 108638) has provided contrast media with reduced osmotoxicity allowing contrast effective iodine concentration to be achieved with hypotonic solution, and has even allowed correction of ionic imbalance by inclusion of plasma ions while still maintaining the contrast medium Visipaque™ at the desired osmolality (WO 90/01194 and WO 91/13636).

The X-ray contrast media at commercial high iodine concentration have relative high viscosity, ranging from about 15 to about 60 mPas at ambient temperature. Generally, contrast media where the contrast enhancing agent is a dimer has higher viscosity than the corresponding contrast media where the contrast enhancing agent is the monomer corresponding to the dimer. Such high viscosities may pose problems to the administrators of the contrast medium, requiring relatively large bore needles or high applied pressure, and are particularly pronounced in pediatric radiography and in radiographic techniques which require rapid bolus administration, e.g. in angiography.

X-ray contrast agents of high molecular weight has been proposed, e.g. polymers with substituted triiodinated phenyl groups grafted on the polymer, see EP 354836, EP 436316 and U.S. Pat. No. 5,019,370. Further, WO 9501966, EP 782563 and U.S. Pat. No. 5,817,873 read on compounds having e.g. 3 and 4 substituted triiodinated phenyl groups arranged linearly or around a central core. However, none of these proposed compounds are on the market.

Hence there still exists a desire to develop contrast agents that solves one or more of the problems discussed above. Such agents should ideally have improved properties over the soluble iodine containing compounds on the market in one or more of the following properties: renal toxicity, osmolality, viscosity, solubility, injection volumes/iodine concentration and attenuation/radiation dose.

### SUMMARY OF THE INVENTION

The present invention provides compounds useful as contrast media having improved properties over the known media with regards to at least one of the following criteria osmolality (and hence the renal toxicity), viscosity, iodine concentration and solubility. The contrast media comprises iodine containing contrast enhancing compounds where iodine containing compounds are chemical compounds containing a central aliphatic N-heterocyclic central moiety such as pyrrolidine or piperidine heterocycles allowing for the arrangement of three iodinated phenyl groups bound thereto through amide linker groups. The iodine containing contrast enhancing compounds can be synthesized from commercially available and relatively inexpensive starting materials.

### DETAILED DESCRIPTION OF THE INVENTION

The new compounds of the invention, their use as X-ray contrast agents, their formulation and production are specified in the claims below and in the specification.

The contrast enhancing compounds are synthetic chemical compounds of formula (I)

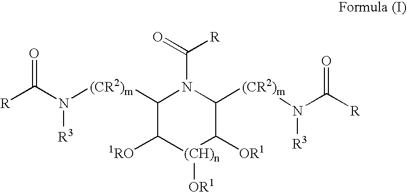

wherein
each R¹ independently are the same or different and denote a hydrogen atom or a C₁-C₄ alkyl group where the alkyl group may be substituted by hydroxyl groups and interrupted by an oxygen atom;
each R² independently are the same or different and denote a hydrogen atom, a hydroxyl group or a C₁-C₄ alkyl group where the alkyl group may be substituted by hydroxyl groups and interrupted by an oxygen atom;
each R³ independently are the same or different and denote a hydrogen atom or a C₁-C₄ alkyl group where the alkyl group may be substituted by hydroxyl groups and interrupted by an oxygen atom;
m is an integer from 1 to 4
n is a integer of 0 or 1; and
each R independently are the same or different and denote a triiodinated phenyl group, preferably a 2,4,6-triiodinated phenyl group further substituted by two groups R⁴ wherein each R⁴ are the same or different and denote a hydrogen atom or a non-ionic hydrophilic moiety, provided that at least one R⁴ group in the compound of formula (I) is a hydrophilic moiety.
and salts or optical active isomers thereof.

The substituents R¹ above are the same or different. Preferably the R¹ groups denote hydrogen atoms or C₁-C₄ alkyl groups. More preferably all R¹ are the same and denote methyl groups or hydrogen atoms, and specifically hydrogen atoms.

It is further preferred that the substituents R² denote hydrogen atoms or methyl groups and preferably all R² groups are the same. Most preferred each of the R² groups denotes a hydrogen atom.

The substituents R³ each preferably denote hydrogen atoms or methyl groups and preferably both R³ groups are the same. Most preferred each of the R³ groups denotes a hydrogen atom.

Both m are preferably the same and denote the integer of 1 or 2, most preferred they are both 1.

When n denotes 0, the central ring will be a 3,4 dihydroxy- or dialkoxy-substituted pyrrolidine ring system, further substituted in the remaining 1,2 and 5 positions with iodinated phenyl groups R bound to the heterocyclic ring by linker groups.

Likewise, when n denotes 1, the central ring will be a 3,4,5-trihydroxy- or trialkoxy-substituted piperidine ring system, further substituted in the remaining 1,2 and 6 positions with iodinated phenyl groups R bound to the heterocyclic ring by linker groups.

Each of the iodinated R groups can be the same or different and preferably denote a 2,4,6-triiodinated phenyl group, further substituted by two groups R⁴ in the remaining 3 and 5 positions in the phenyl moiety.

The non-ionic hydrophilic moieties may be any of the non-ionizing groups conventionally used to enhance water solubility. Hence, the R⁴ substituents may be the same or different and shall preferably all denote a non-ionic hydrophilic moiety comprising esters, amides and amine moieties, optionally further substituted by a straight chain or branched chain C₁₋₁₀ alkyl groups, preferably C₁₋₅ alkyl groups, where the alkyl groups also may have one or more CH₂ or CH moieties replaced by oxygen or nitrogen atoms. The R⁴ substituents may also further contain one or more groups selected from oxo, hydroxyl, amino or carboxyl derivative, and oxo substituted sulphur and phosphorus atoms. Each of the straight or branched alkyl groups preferably contain 1 to 6 hydroxy groups and more preferably 1 to 3 hydroxy groups. Therefore, in a further preferred aspect, the R⁴ substituents are the same or different and are polyhydroxy C₁₋₅ alkyl, hydroxyalkoxyalkyl with 1 to 5 carbon atoms and hydroxypolyalkoxyalkyl with 1 to 5 carbon atoms, and are attached to the iodinated phenyl group via an amide or a carbamoyl linkage.

The R⁴ groups of the formulas listed below are particularly preferred:
—CONH—CH₂—CH₂OH
—CONH—CH₂—CHOH—CH₂OH
—CONH—CH₂—CHOH—CHOH—CH₂OH
—CON(CH₃)CH₂—CHOH—CH₂OH
—CONH—CH—(CH₂OH)₂
—CON—(CH₂—CH₂OH)₂
—CON—(CH₂—CHOH—CH₂—OH)₂
—CONH₂
—CONHCH₃
—CONH—CH₂—CH₂OCH₃
—CONH—OCH₃
—CONH—CH₂—CHOH—CH₂OCH₃
—CON(CH₂—CHOH—CH₂OH)(CH₂—CH₂OH)
—CONH—C(CH₂OH)₃
—CONH—CH(CH₂OH)(CHOH—CH₂OH)
—CONH—CHOCH₃—CH₂OH
—CONH—C(CH₂OH)₂CH₃
—NHCOCH₂OH
—N(COCH₃)H
—N(COCH₃) C₁₋₃ alkyl
—N(COCH₃)— mono, bis or tris-hydroxy C₁₋₄ alkyl
—N(COCH₂OH)— hydrogen, mono, bis or tris-hydroxy C₁₋₄ alkyl
—N(CO—CHOH—CH₂OH)— hydrogen, mono, bis or trihydroxylated C₁₋₄ alkyl
—N(CO—CHOH—CHOH—CH₂OH)— hydrogen, mono, bis or trihydroxylated C₁₋₄ alkyl
—N(COCH—(CH₂OH)₂)— hydrogen, mono, bis or trihydroxylated C₁₋₄ alkyl, and
—N(COCH₂OH)₂

Even more preferably the R⁴ groups will be equal or different and denote one or more moieties of the formulas —CONH—CH₂—CHOH—CH₂—OH, —CONHCH₂—CHOH—CHOH—CH₂OH, —CON(CH₃)CH₂—CHOH—CH₂OH, —CONH—CH—(CH₂—OH)₂, —CONH—C(CH₃)(CH₂CH₂OH), —CON—(CH₂—CH₂—OH)₂, —CON—(CH₂—CHOH—CH₂—OH)₂, —CONH—CH₂—CHOH—CH₂—OH, —NHCOCH₂OH, —NHCO—CHOH—CH₂OH, —NHCO—CHOH—CHOH—CH₂OH and —N(COCH₂OH)— mono, bis or tris-hydroxy C₁₋₄ alkyl, and even more preferably all R groups are the same and the R⁴ groups in each R are different and denote —CONH—CH₂—CHOH—CH₂—OH, —CON(CH₃)CH₂—CHOH—CH₂OH, —CON—(CH₂—CHOH—CH₂—OH)₂, —NHCO—CHOH—CH₂OH, —NHCO—CHOH—CHOH—CH₂OH and —NHCOCH₂OH.

Thus, preferred structures according to the invention include the compounds of formulas (IIa) and (IIb):

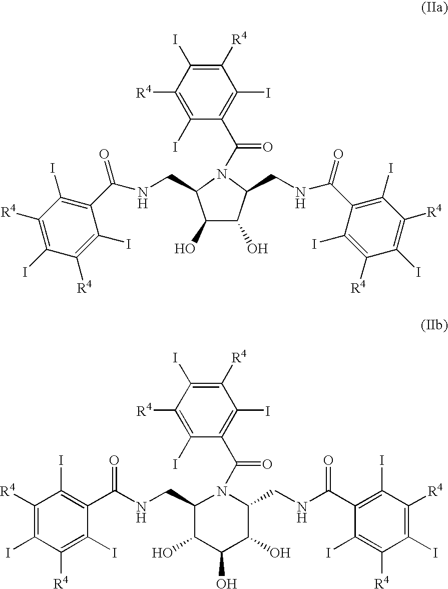

In formulas (IIa) and (IIb), each group R⁴ has the meaning above, more preferably each iodophenyl group R are the same and the R⁴ groups all denote non-ionic hydrophilic moieties.

Further preferred examples the structures according to the invention is represented by formulas (IIIa), (IIIb) and (IIIc):

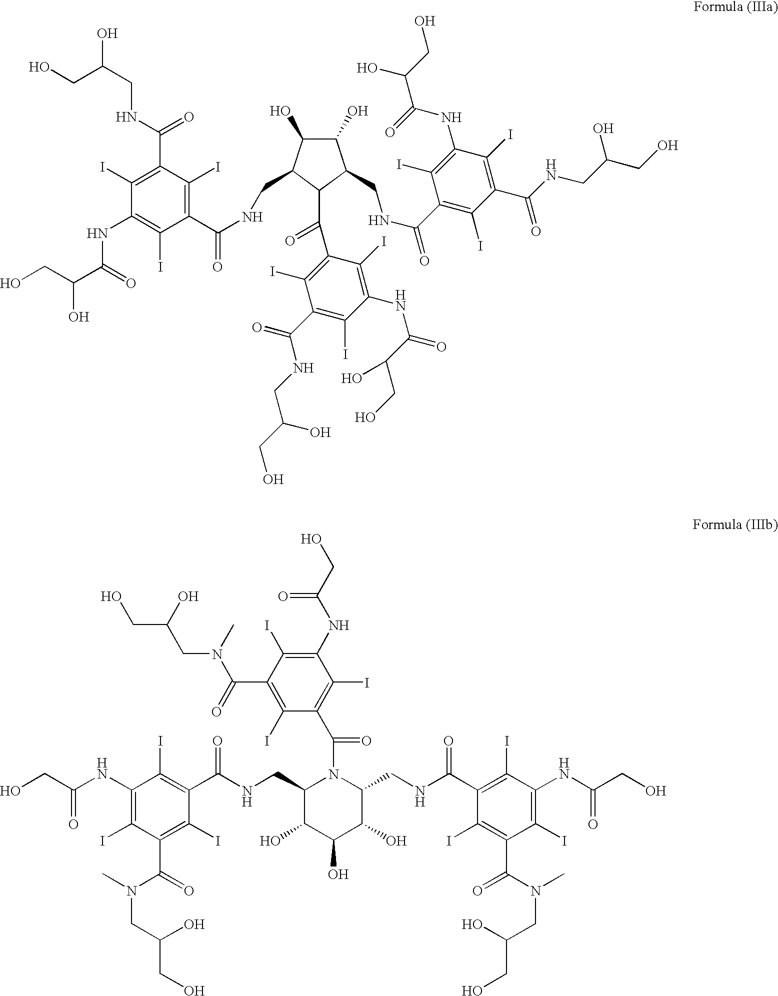

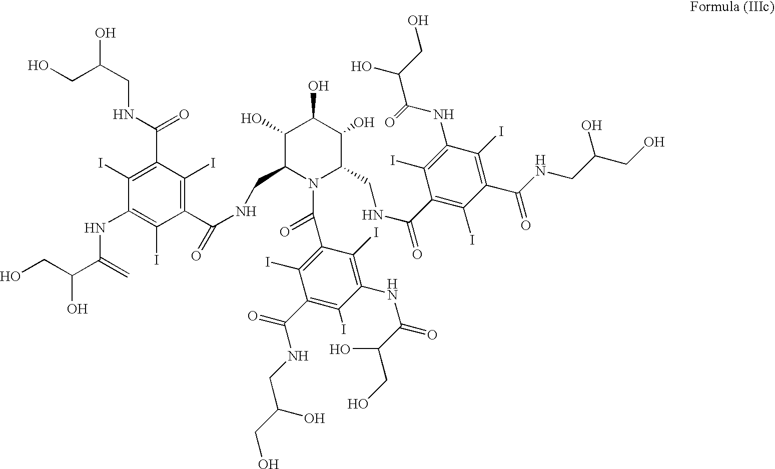

Compounds containing three iodinated phenyl groups are large molecules that are not easily soluble in polar solvents such as water. The hydrophilic substituents R⁴ will increase the solubility of the compounds. Adding polar substituents to the central ring system and breaking the symmetry of the molecule will enhance the solubility of the final product of formula (I) further.

The compounds of formula (I) will attain a relatively compact, folded conformation. Such conformation are relatively round and globular form such as a star-form with the relatively bulky iodinated phenyl substituents filling up the area between the 3 arms of the star or a “stacked spoon” form where the iodinated phenyl groups are aligned as the spoon “bowls” in a stack of spoons. Globular molecules will usually have enhanced solubility compared with similar molecules with a more planar structure and also have lower viscosities.

At an iodine concentration of 320 mg/ml, which is a common concentration for commercially available iodinated contrast media, the concentration of the compound of formula (I) will be approximately 0.28 M (Molar). The contrast medium will also be hypoosmolar at this iodine concentration, and this is an advantageous property with regards to the nephrotoxicity of the contrast medium. It is also possible to add electrolytes to the contrast medium to lower the cardiovascular effects as explained in WO 90/01194 and WO 91/13636.

Compounds of formula (I) also comprises optical active isomers. Both enantiomerically pure products as well as mixtures of optical isomers are included.

The compounds of the invention may be used as contrast agents and may be formulated with conventional carriers and excipients to produce diagnostic contrast media.

Thus viewed from a further aspect the invention provides a diagnostic composition comprising a compound of formula (I) as described above together with at least one physiologically tolerable carrier or excipient, e.g. in aqueous solution for injection optionally together with added plasma ions or dissolved oxygen.

The contrast agent composition of the invention may be in a ready to use concentration or may be a concentrate form for dilution prior to administration. Generally compositions in a ready to use form will have iodine concentrations of at least 100 mg l/ml, preferably at least 150 mg l/ml, with concentrations of at least 300 mg l/ml, e.g. 320 mg l/ml being preferred. The higher the iodine concentration, the higher is the diagnostic value in the form of X-ray attenuation of the contrast media. However, the higher the iodine concentration the higher is the viscosity and the osmolality of the composition. Normally the maximum iodine concentration for a given contrast media will be determined by the solubility of the contrast enhancing agent, e.g. the iodinated compound, and the tolerable limits for viscosity and osmolality.

For contrast media which are administered by injection or infusion, the desired upper limit for the solution's viscosity at ambient temperature (20° C.) is about 30 mPas, however viscosities of up to 50 to 60 mPas and even more than 60 mPas can be tolerated. For contrast media given by bolus injection, e.g. in angiographic procedures, osmotoxic effects must be considered and preferably the osmolality should be below 1 Osm/kg H₂O, preferably below 850 mOsm/kg H₂O and more preferably about 300 mOsm/kg H₂O.

With the compounds of the invention such viscosity, osmolality and iodine concentrations targets can be met. Indeed, effective iodine concentrations can be reached with hypotonic solutions. It may thus be desirable to make up the solution's tonicity by the addition of plasma cations so as to reduce the toxicity contribution that derives from the imbalance effects following bolus injection. Such cations will desirably be included in the ranges suggested in WO 90/01194 and WO 91/13636.

In particular, addition of sodium and calcium ions to provide a contrast medium isotonic with blood for all iodine concentrations is desirable and obtainable. The plasma cations may be provided in the form of salts with physiologically tolerable counterions, e.g. chloride, sulphate, phosphate, hydrogen carbonate etc., with plasma anions preferably being used.

In a further embodiment the invention provides diagnostic agents comprising a compound of formula (I) and diagnostic compositions comprising a compound of formula (I) together with pharmaceutically acceptable carriers or excipients. The diagnostic agents and compositions are preferably for use in X-ray diagnosis.

The contrast media containing compounds of formula (I) can be administered by injection or infusion, e.g. by intervascular administration. Alternatively, contrast media containing compounds of formula (I) may also be administered orally. For oral administration the contrast medium may be in the form of a capsule, tablet or as liquid solution

Hence, the invention further embraces use of a diagnostic agent and a diagnostic composition containing a compound of formula (I) in X-ray contrast examinations and use of a compound of formula (I) for the manufacture of a diagnostic composition for use as an X-ray contrast agent.

A method of diagnosis comprising administration of compounds of formula (I) to the human or animal body, examining the body with a diagnostic device and compiling data from the examination is also provided. In the method of diagnosis the body may also be preadministrated with compounds of formula (I).

Furthermore, a method of imaging, specifically X-ray imaging is provided, which comprises administration of compounds of formula (I) to the human or animal body, examining the body with a diagnostic device and compiling data from the examination and optionally analysing the data. In the method of imaging the body may also be preadministrated with compounds of formula (I).

### EXAMPLES

The invention is further described in the following examples, which are in no way intended to limit the scope of the invention.

### General Preparation

The compounds of the general formula (I) can be synthesized by multistep procedures from starting materials that are either known from the state of art or that are commercially available. Tri-iodinated phenyl groups R and precursors thereof are commercially available or can be produced following procedures described or referred to e.g. in WO95/35122 and WO98/52911. 5-amino-2,4,6-triiodo-isophtalic acid for example is available e.g. from Aldrich and 5-amino-2,4,6-triiodo-N,N′-bis(2,3-dihydroxypropyl)-isophtalamide is commercially available e.g. from Fuji Chemical Industries, Ltd.

By way of example, the compound of formula (IIa) is produced according to the following procedure:

Preparation of the Pyrrolidine Derivative (IIa) can be Carried Out as Shown in the schemes below. Using the procedure described by Xumu Zhang et al., J. Org. Chem, 2000, 65, 3489-3495, the commercially available D-Mannitol could be transformed in three steps to the corresponding di-benzyl-D-mannitol (a). The tetrol (a) can easily be transformed into the corresponding diazidodiol (b) by sodium azide substitution of the corresponding primary ditosylate. When reacted with triphenylphosphine the diazidodiol (b) give the N—H bis-aziridine which when treated with (Boc)₂O should lead to the N-Boc bis-aziridine (c). Sodium azide nucleophilic ring opening of (c) followed by deprotection will lead to the pyrrolidine (d)

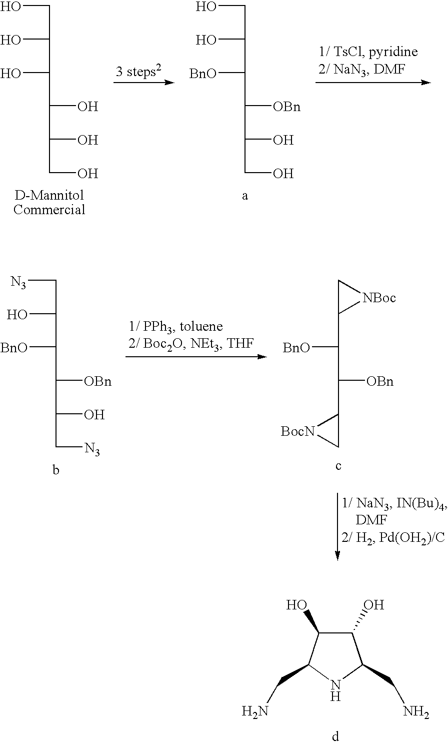

The pyrrolidone derivative (d) can alternatively be prepared according to the reactions described in Preparations A) and B) below.

The pyrrolidine (d) will be reacted with the acyl chlorides such as (e) to form trimers derivatives similar to the trimer (IIa)

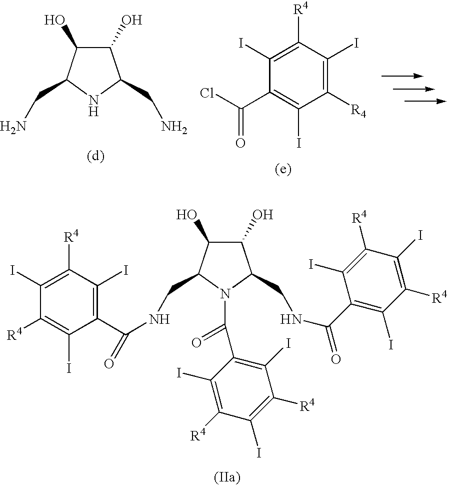

Preparation of the piperidine derivatives (6) can be carried as illustrated in the schemes below.

Using the procedure reported by W. R. Kobertz, C. R. Bertozzi, M. D. Bednarski, J. Org. Chem., 1996, 61, 1894-1897, the commercially available tetra-O-benzyl-α-D-glucopyranoside could be transformed in four steps to the corresponding 6-azido-α-D-glucopyranoside (f). Acid hydrolysis followed by a reduction using NaBH₄, easily gives access to the diol (g). Compound (g) could then be treated with PPh₃ to form the corresponding aziridine which can be protected in-situ with Boc₂O to get the aziridine (h). The alcohol (h) is oxidised and then submitted to a diastereoselective Strecker reaction to form compound (i). A reflux in EtOH in the presence of DIPEA gives access to the imino sugar (j) which is then deprotected to get the piperidine (k).

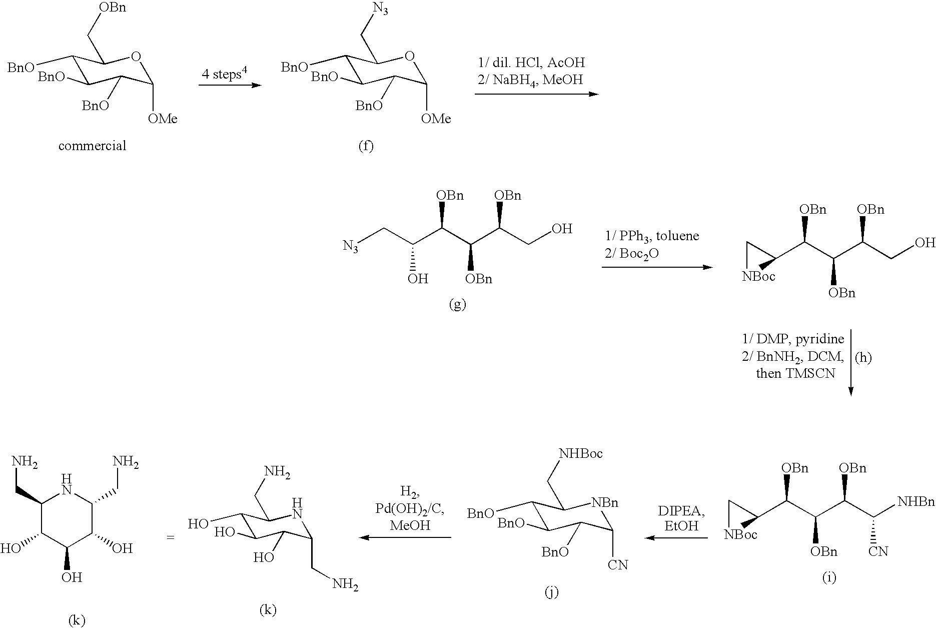

The piperidine derivative of formula (IIb) can also be prepared according to preparation C) below.

The piperidine (k) will be reacted with the acyl chlorides such as (e) to form trimer derivatives similar to the trimer (IIb)

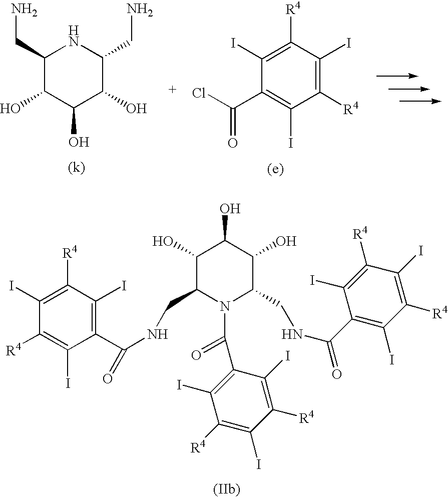

### Preparation of Intermediates:

### Preparation A)

### (2R,3S,4S,5S)-2-Aminomethyl-3,4-bis-benzyloxy-5-(tert-butoxycarbonylamino-methyl)-pyrrolidine-1-carboxylic acid tert-butyl ester (1)

The synthesis of (2R,3S,4S,5S)-2-Aminomethyl-3,4-bis-benzyloxy-5-(tert-butoxycarbonylamino-methyl)-pyrrolidine-1-carboxylic acid tert-butyl ester (1) is described in Tetrahedron Lett. 1995; 36; 44; 8015-8018 and references therein.

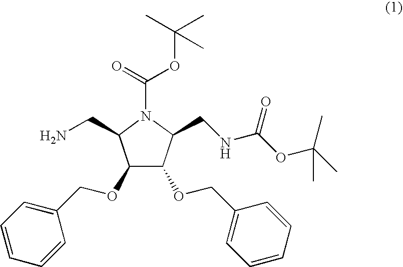

### Preparation B)

### (2R,3S,4S,5S)-2,5-Bis-aminomethyl-pyrrolidine-3,4-diol (2)

(2R,3S,4S,5S)-2-Aminomethyl-3,4-bis-benzyloxy-5-(tert-butoxycarbonylamino-methyl)-pyrrolidine-1-carboxylic acid tert-butyl ester (1) is dissolved in ethanol and placed under a hydrogen atmosphere in the presence of 10 mol % Pd/C at RT and the reaction is monitored by LCMS. On completion the reaction mixture is filtered and concentrated. The crude intermediate is dissolved in DCM and treated with TFA. The solvent is removed under reduced pressure to give (2R,3S,4S,5S)-2,5-Bis-aminomethyl-pyrrolidine-3,4-diol (2).

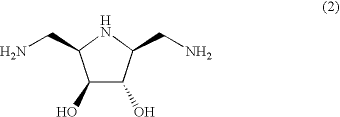

### Preparation C)

### (2R,3S,4R,5R,6R)-2,6-Bis-aminomethyl-piperidine-3,4,5-triol (3)

The synthesis of (2R,3S,4R,5R,6R)-2,6-Bis-aminomethyl-piperidine-3,4,5-triol (3) is described in Tetrahedron Lett. 1997; 38; 51; 8899-8902 and references therein.

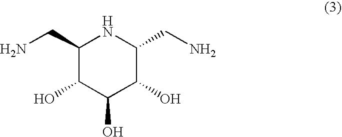

### Preparation of N-Acetylated Monoamides Derivatives

5-amino-2,4,6-triiodo-isophtalic acid available from Aldrich is treated with thionyl chloride to form the corresponding 5-Amino-2,4,6-triiodo-isophthaloyl dichloride. 5-Amino-2,4,6-triiodo-isophthaloyl dichloride is next reacted with either allylamine, N-methyl allylamine or N,N-diallylamine to form respectively 3-Allylcarbamoyl-5-amino-2,4,6-triiodo-benzoyl chloride, 3-(Allyl-methyl-carbamoyl)-5-amino-2,4,6-triiodo-benzoyl chloride and 3-Amino-5-diallylcarbamoyl-2,4,6-triiodo-benzoyl chloride. The mono amides is then reacted with either acetoxyacetyl chloride commercially available from Aldrich, 2,3-diacetoxypropanoyl chloride or 2,3-diacetoxypropanoyl chloride to form the desired N acetylated derivatives. These procedures are further illustrated in the preparations below.

### Preparation D)

### Synthesis of 5-Amino-2,4,6-triiodo-isophthaloyl dichloride (4)

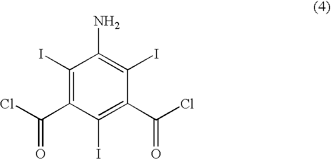

5-Amino-2,4,6-triiodo-isophtalic acid (30 g, 0.054 mol) (commercially available from Aldrich), thionyl chloride (8.2 ml, 0.113 mol) and pyridine (0.2 ml) in 1,2 dichloroethane (20 ml) were heated to 70° C. A portion of thionyl chloride (15.2 ml, 0.21 mol) was added dropwise during 1½ to 2 hrs, and the mixture was heated to 85° C. for 6 hrs. After cooling the reaction mixture to room temperature, it was poured into 300 g of ice-water. The yellow precipitate that formed was filtered off, sucked dry and then washed with water until washings showed a pH of ca 5. The filter cake was then dried in a vacuum oven at 50° C. for 3 hrs. A light yellow powder was obtained 31 g (˜quant.) as the desired product.

¹³C NMR (DMSOd₆) 66, 78.4, 148.9, 149.2, 169

MS (ES−) found 593.5 [M+H+], expected 593.7

### Preparation E)

### Synthesis of 3-Allylcarbamoyl-5-amino-2,4,6-triiodo-benzoyl chloride (5)

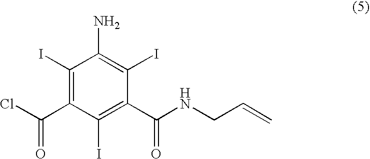

5-Amino-2,4,6-triiodo-isophthaloyl dichloride (4) (100 g, 168 mmol) was dissolved in anhydrous THF (400 ml), the allylamine was dissolved in 100 ml THF, and added dropwise to the solution over 2.5 hours. The mixture was heated to 50 deg C. and stirred overnight under a nitrogen atmosphere. The reaction was monitored by TLC (2% MeOH in DCM) on silica gel plates, bis-acid chloride had an R_{f} of ˜0.9, the monoallylamide ˜0.75 and the bis-allylamide ˜0.25. Once the reaction was deemed complete, the solution was filtered, vacuumed to dryness, then dissolved in 500 ml of ethyl acetate this solution was then loaded onto silica and purified on a 750 g column using ethyl acetate (B) and petrol (A) (10%→100% B over ˜10 column volumes). The pure fractions were collected and identified by TLC, the desired fractions were then vacuumed to dryness. The structure was confirmed by ¹H and ¹³C NMR and purity by LCMS.

### Preparation F)

### Synthesis of 3-(Allyl-methyl-carbamoyl)-5-amino-2,4,6-triiodo-benzoyl chloride (6)

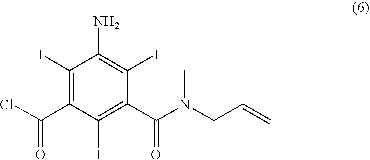

Typically 5-amino-2,4,6,triiodoisophthaloyl dichloride (4) (100 g, 20 mmol) was dissolved in anhydrous THF (500 ml), the N-methyl allylamine (25 ml) was dissolved in 50 ml THF, and added dropwise to the solution over 1 hour. The mixture was heated to 50 deg C. and stirred overnight. The crude mixture was analysed by LCMS and this confirmed that the reaction mixture contained the desired product, ‘bis-acid chloride’ and ‘bis-N-methyl-allylamide’. The reaction was also monitored by TLC (2% MeOH in DCM) on silica gel plates, bis-acid chloride had an R_{f} of ˜0.98, the mono-N-methylallylamide ˜0.73 and the bis-N-methylallylamide ˜0.25. Once the reaction was deemed complete, the solution was filtered, vacuumed to dryness, then dissolved in 500 ml of ethyl acetate this solution was then loaded onto silica and purified on a 750 g column using ethyl acetate (B) and petrol (A) (10%→100% B over ˜10 column volumes). The pure fractions were collected and identified by TLC, the desired fractions were then vacuumed to dryness. The structure was confirmed by ¹H and ¹³C NMR and purity by LCMS.

### Preparation G)

### Synthesis of 3-Amino-5-diallylcarbamoyl-2,4,6-triiodo-benzoyl chloride (7)

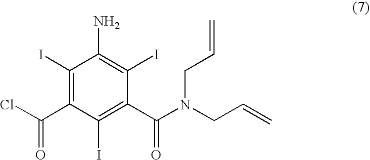

The Bis acid chloride (4) (50 g, 84 mmol) was dissolved in anhydrous THF (200 ml), the N,N′-di-allylamine (21 ml, 168 mmol) was dissolved in 50 ml THF, and added dropwise to the solution over 1 hour. The mixture was heated to 50 deg C. and stirred overnight. The crude mixture was analysed by LCMS and this confirmed that the reaction mixture contained the desired product, bis-acid chloride’ and ‘bis-N-diallylamide’. Once the reaction was deemed complete, the solution was filtered, vacuumed to dryness, then dissolved in 500 ml of ethyl acetate this solution is then loaded onto silica and purified on a 750 g column using ethyl acetate (B) and petrol (A) (10%→100% B over ˜10 column volumes). The pure fractions are collected and identified by TLC, the desired fractions are then vacuumed to dryness. The structure was confirmed by ¹H NMR and purity by LCMS (656.82 (+ve))

### Preparation H)

### Synthesis of acetic acid (3-allylcarbamoyl-5-chlorocarbonyl-2,4,6-triiodo-phenylcarbamoyl)-methyl ester (8)

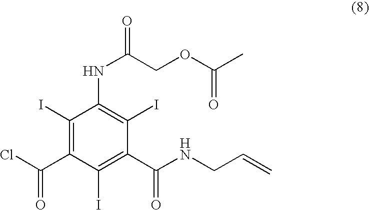

3-Allylcarbamoyl-5-amino-2,4,6-triiodo-benzoyl chloride (5) (5 g, 8.11 mmol) was dissolved in dry DMA (5 mL) and acetoxyacetylchloride (1.73 mL, 16.2 mmol) was added. The reaction was stirred overnight at room temperature with nitrogen bubbling through. The reaction mixture was diluted with ethyl acetate (100 mL) and washed with ice-water (5×20 mL). The organics were collected, dried over MgSO₄, filtered and evaporated to dryness under reduced pressure. The residue was washed with acetonitrile, filtered and dried under vacuum to give acetic acid (3-allylcarbamoyl-5-chlorocarbonyl-2,4,6-triiodo-phenylcarbamoyl)-methyl ester as a white solid. (4.47 g, 77%). The structure was confirmed by ¹H and ¹³C NMR, and purity by LCMS.

### Preparation I)

### Synthesis of acetic acid [3-(allyl-methyl-carbamoyl)-5-chlorocarbonyl-2,4,6-triiodo-phenylcarbamoyl]-methyl ester (9)

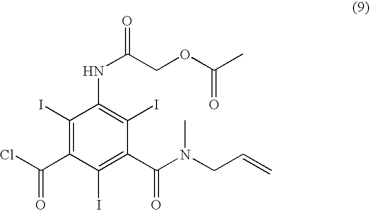

3-(Allyl-methyl-carbamoyl)-5-amino-2,4,6-triiodo-benzoyl chloride (6) (5 g, 7.93 mmol) was dissolved in dry DMA (20 mL) was acetoxyacetyl chloride (1.7 mL, 15.9 mmol) was added dropwise. The reaction mixture was stirred at overnight at RT, with nitrogen bubbling through the reaction mixture. The reaction was monitored by TLC on silica gel plates eluting with ethyl acetate:petrol (1:1). (6) had an Rf of 0.62 and 0.76 whilst there were two new spots at 0.32 and 0.22. The solution was diluted with ethyl acetate (˜100 mL) and washed with ice water/brine (50:50, 20 ml×5). The organics were dried over MgSO₄, filtered, concentrated and dried under high vacuum to give the desired compound (5.26 g, 91%). The structure was confirmed by ¹H and ¹³C NMR, and purity by LCMS.

### Preparation J)

### Synthesis of acetic acid (3-chlorocarbonyl-5-diallylcarbamoyl-2,4,6-triiodo-phenylcarbamoyl)-methyl ester (10)

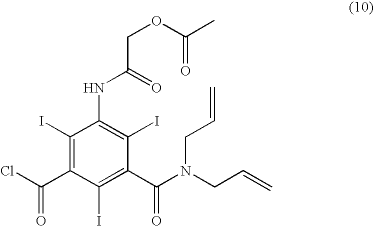

The mono diallylamide (7) (6.56 g, 10 mmol) was dissolved in anhydrous DCM (10 ml), and stirred. The acetoxy acetyl chloride (2.1 ml, 20 mmol) was added to the solution, and heated to 40° C. for 3 days. The solvent was removed at reduced pressure and the reaction mixture was absorbed onto silica gel. The crude mixture was separated by silica gel chromatography 10% EtOAc/Petrol =>100% EtOAc over 11 CVs. The main peak was collected, concentrated at reduced pressure and analysed by both LCMS (m/z 756.83 (+ve) and NMR. This indicated the desired material had been made in good purity. The yield was 6.5 g (86%).

### Preparation K)

### Synthesis of lithium 2,3-dihydroxypropanoate (11)

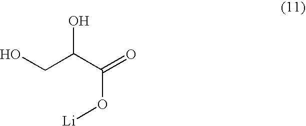

D,L-Serine (115.5 g, 1.10 mole) was added to a mixture of conc. sulfuric acid (75 g) in water (1.25 L) and the mixture was cooled to ca 5° C. Sodium nitrite (68.3 g, 0.99 mole) dissolved in water (500 ml) was added slowly during 3 h while temperature was kept at 5°-10° C. Then sulfuric acid (60 g) dissolved in water (200 ml) and cooled to ca 5° C. in a ice/water mixture, was added. A new portion of sodium nitrite (68.3 g, 0.99 mole) dissolved in water (500 ml) was added slowly during 2 h, while temperature was kept at 5°-10° C. The mixture was stirred at ambient temperature over night and then concentrated to a volume of ca 700 ml. Lithium hydroxide (22.7 g, 0.95 mole), dissolved in water (100 ml) was added. The mixture was now poured into a stirred mixture of methanol (1 L) and acetone (0.3 L). The precipitate formed was filtered off and washed with methanol/acetone (1/0.3 100 ml). The combined filtrates were now evaporated to a small volume (ca. 300 ml) and pH was adjusted to 7 by addition of a 5M solution of lithium hydroxide (ca. 200 ml). The mixture was evaporated to dryness and abs. ethanol (600 ml) was added, the product dissolved by heating and the mixture evaporated to dryness. The residue was then co evaporated twice with toluene (2×300 ml), and pumped in vacuo. There was of a gum like product 130 g. Identity was checked by ¹H NMR in D₂O.

### Preparation L)

### Synthesis of 2,3-diacetoxypropanoic acid (12)

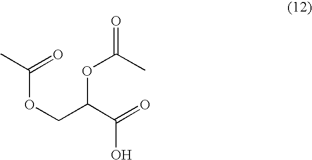

Acetyl chloride (500 ml) was added dropwise without stirring to the gummy like mass of lithium 2,3-dihyroxypropanoate (11) (171 g, 1.51 mole). The gummy like mass dissolved slowly and the mixture was left for 24 h at ambient temperature. Then the mixture was stirred and heated to reflux for 6 h. After cooling the mixture was diluted with ethyl acetate (700 ml) and filtered through a tight glass filter (por. G4). The filtrate was evaporated to a oil, which was dissolved in ethyl acetate (750 ml) and washed with water (2×70 ml, pH=2). After drying over magnesium sulfate and treatment with activated charcoal (1.5 g) the mixture was filtered. The filtrate was evaporated to a light orange coloured oil. Yield (crude) 218 g (75%). Purity checked by ¹HNMR in CDCl₃.

### Preparation M)

### Synthesis of 2,3-diacetoxypropanoyl chloride (13)

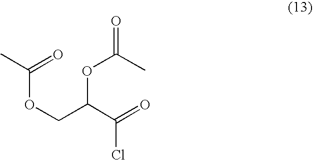

Thionyl chloride (62 ml, 0.86 mole) was added dropwise to 2,3-diacetoxypropanoic acid (12) in a flask to which a drop of N,N-dimethylformamide had been added. The mixture was then stirred at ambient temperature over night and then evaporated to a syrup at a temperature ≦40° C. The syrup was taken up in diethyl ether (60 ml) and activated charcoal (0.3 g) added. The mixture was then filtered through a tight glass filter and evaporated in vacuo (10 torr). The oily residue was distilled in a Kugelrohr apparatus to give 24.6 g (68%). Identity and purity checked by ¹HNMR in CDCl₃.

### Preparation N)

### Synthesis of acetic acid 2-acetoxy-2-(3-allylcarbamoyl-5-chlorocarbonyl-2,4,6-triiodo-phenylcarbamoyl)-ethyl ester (14)

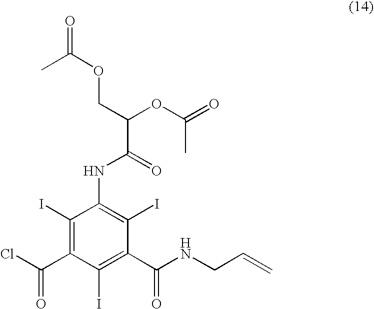

In dry three necked round bottom flask fitted with an additional funnel was poured 5-amino-2,4,6-triiodoisophtalic-3-allyamide-benzoyl chloride (5) (10 g, 0.016 mol) and 10 ml of DMAC. To the stirred and cooled solution of 2,3-diacetoxypropanoyl chloride (13) (6.8 g, 0.032 mol) in 10 ml of DMAc was added dropwise over 15-20 minutes. The reaction was allowed to react 20 hours with a gentle flow of nitrogen bubbling through the reaction. The solvent was concentrated under vacuo and the resulting dark brown crude mixture was purified via normal phase column chromatography eluting with ethyl acetate and petroleum ether. After purification 11 g of an off-white solid was obtained (90% yield and 98% HPLC purity) Mass found: (ES+) 789, 811 (Na+) and 1576.64, (ES−) 787, 1574

### Preparation O)

### Synthesis of acetic acid 2-acetoxy-2-[3-(allyl-methyl-carbamoyl)-5-chlorocarbonyl-2,4,6-triiodo-phenylcarbamoyl]-ethyl ester (15)

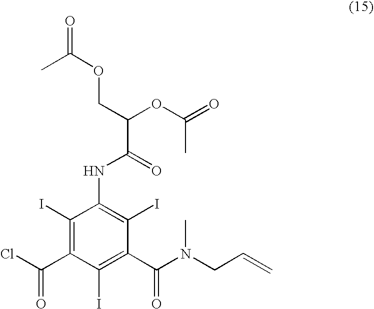

3-(Allyl-methyl-carbamoyl)-5-amino-2,4,6-triiodo-benzoyl chloride (6) (0.19 mol, 120 g) was dissolved in dry N,N-dimethyl acetamide (DMA) (480 ml) and the acid chloride (13) (0.38 ml, 79 g) was added dropwise. The clear yellow red reaction mixture was stirred at overnight at ambient temperature, with nitrogen bubbling through the reaction mixture. The reaction was monitored by TLC on silica gel plates eluting with ethyl acetate:petrol (1:1). After 19 hours the reaction was stopped and the brown solution was diluted with ethyl acetate (˜2.4 L) and washed with ice water/brine (50:50, 480 ml×5). The filtrate was washed again with ethyl acetate. 500 ml of filtrate washed twice with 250 ml of ethyl acetate. The brown solution was poured into a 6 L separating funnel and treated with 200 ml of cold water/brine (1:1) solution. The organics were dried over MgSO₄, filtered and concentrated. The brown oil obtained was dried under high vacuum over night and analysed via LCMS. One major peak was observed with a mass of 803 (M+H⁺) and a purity of 86%. ¹H NMR was carried out (CDCl₃). The NMR spectrum showed residual ethyl acetate. The brown oil was left under high vacuum at 40° C. for 1 hour and then left over night under high vacuum at ambient temperature. The mixture was dissolved in ethyl acetate and supported onto silica gel and purified by silica gel chromatography eluting with ethyl acetate/petrol. The off white solid was dried over night under high vacuum at room temperature and this gave a yield of 56%. LCMS was carried out Luna C18 250×4.6 10 u. Purity 95%, ¹H NMR (CDCl₃) confirmed structure of the desired compound.

### Preparation P)

### Synthesis of acetic acid 2,3-diacetoxy-3-chlorocarbonyl-propyl ester (16)

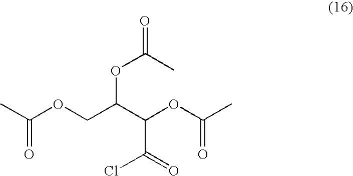

The 2,3,4-triacetoxy-butyric acid (25 g, 0.095 mol) was stirred in thionyl chloride (15.3 mL) at room temperature with a condenser fitted. The reaction was stirred for 48 hours and then the thionyl chloride was removed under reduced pressure to give an oil which was the desire material (26.1 g, 98%).

### Preparation Q)

### Synthesis of acetic acid 2,3-diacetoxy-1-(3-allylcarbamoyl-5-chlorocarbonyl-2,4,6-triiodo-phenylcarbamoyl)-propyl ester (17)

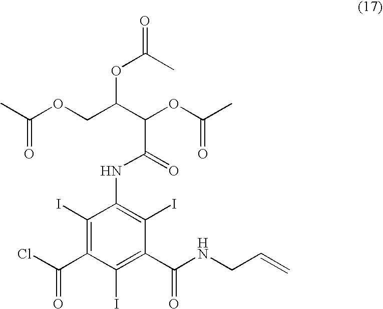

3-Allylcarbamoyl-5-amino-2,4,6-triiodo-benzoyl chloride (5) (20 g, 32.4 mmol) was dissolved in dry DMA (50 mL) and threonic acid chloride triacetate (16) (18.22 g, 64.8 mmol) was added. The reaction was stirred for 3 days at room temperature with nitrogen bubbling through. The reaction mixture was diluted with ethyl acetate (100 mL) and washed with ice-water (5×20 mL). The organics were collected, dried over MgSO₄, filtered and evaporated to dryness under reduced pressure. The solid was adsorbed onto silica gel and purified by column chromatography eluting with DCM: ethyl acetate (0-100%, SiO₂, 750 g, 10 CV) to give acetic acid 2,3-diacetoxy-1-(3-allylcarbamoyl-5-chlorocarbonyl-2,4,6-tri iodo-phenylcarbamoyl)-propyl ester as a yellow solid (15.1 g, 54%).

### Preparation R)

### Synthesis of acetic acid 2,3-diacetoxy-1-[3-(allyl-methyl-carbamoyl)-5-chlorocarbonyl-2,46-triiodo-phenylcarbamoyl]-propyl ester (18)

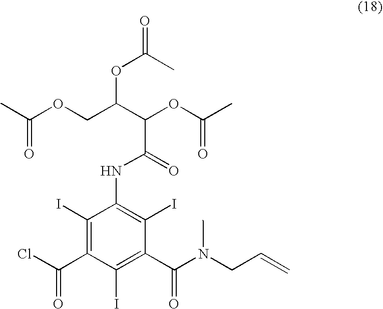

5-amino-2,4,6-triiodoisophathalic mono-N-methyl allylamide (6) (13.5 g, 0.0214 mol) and threonic acid chloride triacetate (16) (11.1 g, 0.0395 mol) were dissolved in dry dimethylacetamide (60 mL) and stirred for 48 hours at room temperature. The reaction mixture was diluted with ethyl acetate (250 mL) and washed with ice-water/brine (50:50, 5×25 mL). The organics were collected and dried over MgSO₄, filtered and evaporated to dryness to give a brown oil. It was purified by column chromatography, eluting with petrol: ethyl acetate (10-100%, 12 column volumes, SiO₂, 330 g) to give the desired product as an off white solid (10.1 g, 54%).

The product was confirmed by ¹H NMR (CDCl₃).

### Preparation of the Trimers:

### (2R,3S,4S,5S)-N, N,N-Tris-N′-(2,3-dihydroxy-propyl)-5-(2-hydroxy-acetylamino)-2,4,6-triiodo-N′-methyl-isophthalamidyl-2,5-Bis-aminomethyl-pyrrolidine-3,4-diol

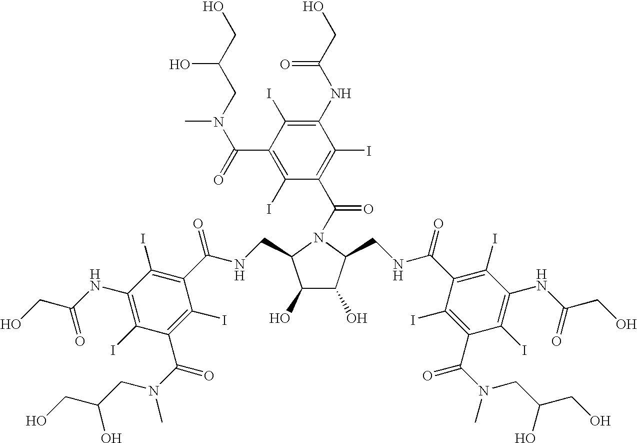

### a) Trimer Formation

To a solution of acetic acid [3-(allyl-methyl-carbamoyl)-5-chlorocarbonyl-2,4,6-triiodo-phenylcarbamoyl]-methyl ester (5) in DMA is added 0.33 equivalent of (2R,3S,4S,5S)-2,5-Bis-aminomethyl-pyrrolidine-3,4-diol (2) and 0.33 equivalent of triethylamine. The reaction is stirred at ambient temperature until the reaction proceeds no further. The reaction mixture is extracted into ethyl acetate and washed with water to remove the DMA. The organic layer is dried over MgSO₄. Silica gel chromatography is used to separate the products of the reaction to yield the desired trimer.

Same methodology is applied to trimers derived from (2R,3S,4R,5R,6R)-2,6-Bis-aminomethyl-piperidine-3,4,5-triol.

### b) Cis-Dihydroxylation

The trimer is dissolved in the minimum of acetone/water (9:1) and treated with 1 ml of a solution of osmium catalyst (1.0 g OsO₄, 100 ml t-BuOH 100 ml and 10 drops of t-BuOOH) and up to 20 equivalents of N-methylmorpholine N-oxide. The reaction is worked up by quenching the reaction with a solution of sodium hydrogen sulphite (15%, 15 ml) the mixture is evaporated to dryness. The crude material is used in the next step without further purification.

### d) Hydrolysis

The crude material from the previous step is dissolved in the minimum amount of methanol and treated with aqueous ammonia. The reaction is stirred at ambient temperature and monitored by LCMS. When complete the reaction mixture is concentrated to dryness, dissolved in the minimum amount of water, filtered and purified by preparative HPLC. The material is characterised by a minimum of NMR and LCMS.

Following the procedure above, the following compounds can be prepared:
(2R,3S,4S,5S)-N,N,N-Tris-N′-(2,3-dihydroxy-propyl)-5-(2-hydroxy-acetylamino)-2,4,6-triiodo-isophthalamidyl-2,5-Bis-aminomethyl-pyrrolidine-3,4-diol
(2R,3S,4S,5S)-N,N,N-Tris-N′,N′-bis-(2,3-dihydroxy-propyl)-5-(2-hydroxy-acetylamino)-2,4,6-triiodo-isophthalamidyl-2,5-Bis-aminomethyl-pyrrolidine-3,4-diol
(2R,3S,4S,5S)-N,N,N-Tris-N′-(2,3-dihydroxy-propyl)-5-(2,3-dihydroxy-propionylamino)-2,4,6-triiodo-N′-methyl-isophthalamidyl-2,5-Bis-aminomethyl-pyrrolidine-3,4-diol
(2R,3S,4S,5S)-N,N,N-Tris-N′-(2,3-dihydroxy-propyl)-5-(2,3-dihydroxy-propionylamino)-2,4,6-triiodo-isophthalamidyl-2,5-Bis-aminomethyl-pyrrolidine-3,4-diol
(2R,3S,4S,5S)-N,N,N-Tris-N′,N′-bis-(2,3-dihydroxy-propyl)-5-(2,3-dihydroxy-propionylamino)-2,4,6-triiodo-N′-methyl-isophthalamidyl-2,5-Bis-aminomethyl-pyrrolidine-3,4-diol
(2R,3S,4S,5S)-N,N,N-Tris-N′-(2,3-dihydroxy-propyl)-5-(2,3,4-trihydroxy-butyrylamino)-2,4,6-triiodo-N′-methyl-isophthalamidyl-2,5-Bis-aminomethyl-pyrrolidine-3,4-diol
(2R,3S,4S,5S)-N,N,N-Tris-N′-(2,3-dihydroxy-propyl)-5-(2,3,4-trihydroxy-butyrylamino)-2,4,6-triiodo-isophthalamidyl-2,5-Bis-aminomethyl-pyrrolidine-3,4-diol
(2R,3S,4S,5S)-N,N,N-Tris-N′,N′-bis-(2,3-dihydroxy-propyl)-5-(2,3,4-trihydroxy-butyrylamino)-2,4,6-triiodo-N′-methyl-isophthalamidyl-2,5-Bis-aminomethyl-pyrrolidine-3,4-diol
(2R,3S,4R,5R,6R)-N,N,N-Tris-N′-(2,3-dihydroxy-propyl)-5-(2-hydroxy-acetylamino)-2,4,6-triiodo-N′-methyl-isophthalamide-2,6-Bis-aminomethyl-piperidine-3,4,5-triol
(2R,3S,4R,5R,6R)-N,N,N-Tris-N′-(2,3-dihydroxy-propyl)-5-(2-hydroxy-acetylamino)-2,4,6-triiodo-isophthalamide-2,6-Bis-aminomethyl-piperidine-3,4,5-triol
(2R,3S,4R,5R,6R)-N,N,N-Tris-N′,N′-bis-(2,3-dihydroxy-propyl)-5-(2-hydroxy-acetylamino)-2,4,6-triiodo-isophthalamide-2,6-Bis-aminomethyl-piperidine-3,4,5-triol
(2R,3S,4R,5R,6R)-N,N,N-Tris-N′-(2,3-dihydroxy-propyl)-5-(2,3-dihydroxy-propionylamino)-2,4,6-triiodo-N′-methyl-isophthalamide-2,6-Bis-aminomethyl-piperidine-3,4,5-triol
(2R,3S,4R,5R,6R)-N,N,N-Tris-N′-(2,3-dihydroxy-propyl)-5-(2,3-dihydroxy-propionylamino)-2,4,6-triiodo-isophthalamide-2,6-Bis-aminomethyl-piperidine-3,4,5-triol
(2R,3S,4R,5R,6R)-N,N,N-Tris-N′,N′-bis-(2,3-dihydroxy-propyl)-5-(2,3-dihydroxy-propionylamino)-2,4,6-triiodo-isophthalamide-2,6-Bis-aminomethyl-piperidine-3,4,5-triol
(2R,3S,4R,5R,6R)-N,N,N-Tris-N′-(2,3-dihydroxy-propyl)-5-(2,3,4-trihydroxy-butyrylamino)-2,4,6-triiodo-N′-methyl-isophthalamide-2,6-Bis-aminomethyl-piperidine-3,4,5-triol
(2R,3S,4R,5R,6R)-N,N,N-Tris-N′-(2,3-dihydroxy-propyl)-5-(2,3,4-trihydroxy-butyrylamino)-2,4,6-triiodo-isophthalamide-2,6-Bis-aminomethyl-piperidine-3,4,5-triol
(2R,3S,4R,5R,6R)-N,N,N-Tris-N′,N′-bis-(2,3-dihydroxy-propyl)-5-(2,3,4-trihydroxy-butyrylamino)-2,4,6-triiodo-isophthalamide-2,6-Bis-aminomethyl-piperidine-3,4,5-triol.

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and structures. Such modifications are intended to fall within the scope of the appended claims.

Various publications and patent applications are cited herein, the disclosures of which are incorporated by reference in their entireties.

What is claimed is:

1. Compound of formula (I) 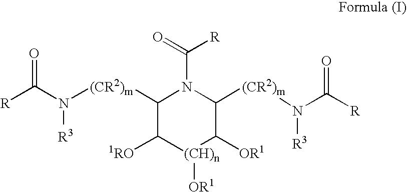
and salts or optical active isomers thereof,
wherein
each R¹ independently is the same or different and denotes a hydrogen atom or a C₁-C₄ alkyl group where the alkyl group may be substituted by hydroxyl groups and interrupted by an oxygen atom;
each R² independently is the same or different and denotes a hydrogen atom, a hydroxyl group or a C₁-C₄ alkyl group where the alkyl group may be substituted by hydroxyl groups and interrupted by an oxygen atom;
each R³ independently is the same or different and denotes a hydrogen atom, a hydroxyl group or a C₁-C₄ alkyl group where the alkyl group may be interrupted by an oxygen atom;
m is an integer from 1 to 4
n is a integer of 0 or 1; and
each R independently is the same or different and denotes a triiodinated phenyl group, preferably a 2,4,6-triiodinated phenyl group further substituted by two groups R⁴ wherein each R⁴ are the same or different and denote a hydrogen atom or a non-ionic hydrophilic moiety, provided that at least one R⁴ group in the compound of formula (I) is a hydrophilic moiety.

2. The compound as claimed in claim 1 wherein each R¹ independently denote a hydrogen atom or a C₁-C₄ alkyl group.

3. The compound as claimed in claim 2 wherein all R¹ are the same and denote methyl groups or hydrogen atoms, preferably a hydrogen atoms.

4. The compound as claimed in claim 1 wherein each R² independently denotes a hydrogen atom or a methyl group.

5. The compound as claimed in claim 4 wherein each R² group denotes a hydrogen atom.

6. The compound as claimed in claim 1 wherein each R³ independently denotes a hydrogen atom or a methyl group.

7. The compound as claimed in claim 6 wherein each R³ group denote a hydrogen atom.

8. The compound as claimed in claim 1 wherein m is the same or different and denotes the integer of 1 or 2, preferably both are 1.

9. The compound as claimed in claim 1 wherein each R is the same or different and denotes a 2,4,6 triiodinated phenyl group, further substituted by two R⁴. groups.

10. The compound as claimed in claim 9 wherein each R⁴ is the same or different and denotes a non-ionic hydrophilic moiety comprising esters, amides and amine moieties, optionally further substituted by a straight chain or branched chain C₁₋₁₀ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

11. The compound as claimed in claim 10 wherein each R⁴ is the same or different and denotes a non-ionic hydrophilic moiety comprising esters, amides and amine moieties, optionally further substituted by a straight chain or branched chain C₁₋₅ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

12. The compound as claimed in claim 10 wherein each R⁴ is the same or different and denotes a non-ionic hydrophilic moiety comprising esters, amides and amine moieties, further substituted by a straight chain or branched chain C₁₋₅ alkyl groups substituted by 1 to 3 hydroxy groups.

13. The compound as claimed in claim 11 wherein each R⁴ are the same or different and are polyhydroxy C₁₋₅ alkyl, hydroxyalkoxyalkyl with 1 to 5 carbon atoms and hydroxypolyalkoxyalkyl with 1 to 5 carbon atoms attached to the iodinated phenyl group via an amide or a carbamoyl linkage.

14. The compound as claimed in claim 11 wherein each R⁴ are the same or different and are selected from groups of the formulas
—CONH—CH₂—CH₂OH
—CONH—CH₂—CHOH—CH₂OH
—CONH—CH₂—CHOH—CHOH—CH₂OH
—CON(CH₃)CH₂—CHOH—CH₂OH
—CONH—CH—(CH₂OH)₂
—CON—(CH₂—CH₂OH)₂
—CON—(CH₂—CHOH—CH₂—OH)₂
—CONH₂
—CONHCH₃
—CONH—CH₂—CH₂OCH₃
—CONH—OCH₃
—CONH—CH₂—CHOH—CH₂OCH₃
—CON(CH₂—CHOH—CH₂OH)(CH₂—CH₂OH)
—CONH—C(CH₂OH)₃
—CONH—CH(CH₂OH)(CHOH—CH₂OH)
—CONH—CHOCH₃—CH₂OH
—CONH—C(CH₂OH)₂CH₃
—NHCOCH₂OH
—N(COCH₃)H
—N(COCH₃) C₁₋₃ alkyl
—N(COCH₃)— mono, bis or tris-hydroxy C₁₋₄ alkyl
—N(COCH₂OH)— hydrogen, mono, bis or tris-hydroxy C₁₋₄ alkyl
—N(CO—CHOH—CH₂OH)— hydrogen, mono, bis or trihydroxylated C₁₋₄ alkyl
—N(CO—CHOH—CHOH—CH₂OH)— hydrogen, mono, bis or trihydroxylated C₁₋₄ alkyl
—N(COCH—(CH₂OH)₂)— hydrogen, mono, bis or trihydroxylated C₁₋₄ alky, and
—N(COCH₂OH)₂

15. The compound as claimed in claim 14 wherein each R⁴ is the same or different and are selected from groups of the formulas —CONH—CH₂—CHOH—CH₂—OH, —CONHCH₂—CHOH—CHOH—CH₂OH, —CONH—CH—(CH₂—OH)₂, —CON—(CH₂—CH₂—OH)₂, —CONH—C(CH₃)(CH₂CH₂OH), —CONH—CH₂—CHOH—CH₂—OH, —CON—(CH₂CHOH—CH₂OH)₂—NHCOCH₂OH and —N(COCH₂OH)— mono, bis or tris-hydroxy C₁₋₄ alkyl.

16. The compound as claimed in claim 1 of formula (IIa) 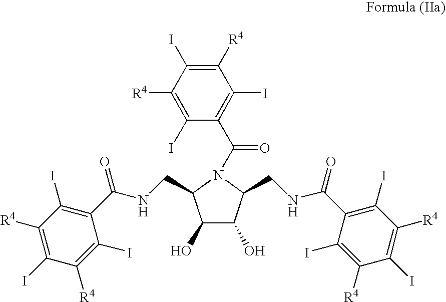
wherein each group R⁴ are as defined in the previous claims, and wherein each iodophenyl groups R is the same and the R⁴ groups all denote non-ionic hydrophilic moieties.

17. The compound as claimed in claim 1 of formula (IIb) 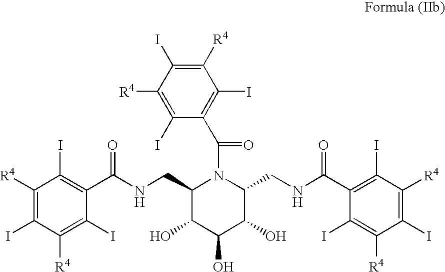
wherein each group R⁴ is as defined in the previous claims, and wherein each iodophenyl groups R are the same and the R⁴ groups all denote non-ionic hydrophilic moieties.

18. The compounds as claimed in claim 1 being
(2R,3S,4S,5S)-N,N,N-Tris-N′-(2,3-dihydroxy-propyl)-5-(2-hydroxy-acetylamino)-2,4,6-triiodo-N′-methyl-isophthalamidyl-2,5-Bis-aminomethyl-pyrrolidine-3,4-diol;
(2R,3S,4R,5R,6R)-N,N,N-Tris-N′-(2,3-dihydroxy-propyl)-5-(2-hydroxy-acetylamino)-2,4,6-triiodo-isophthalamide-2,6-Bis-aminomethyl-piperidine-3,4,5-triol;
(2R,3S,4R,5R,6R)-N,N,N-Tris-N′,N′-bis-(2,3-dihydroxy-propyl)-5-(2-hydroxy-acetylamino)-2,4,6-triiodo-isophthalamide-2,6-Bis-aminomethyl-piperidine-3,4,5-triol;
(2R,3S,4R,5R,6R)-N,N,N-Tris-N′-(2,3-dihydroxy-propyl)-5-(2,3-dihydroxy-propionylamino)-2,4,6-triiodo-N′-methyl-isophthalamide-2,6-Bis-aminomethyl-piperidine-3,4,5-triol;
(2R,3S,4R,5R,6R)-N,N,N-Tris-N′-(2,3-dihydroxy-propyl)-5-(2,3-dihydroxy-propionylamino)-2,4,6-triiodo-isophthalamide-2,6-Bis-aminomethyl-piperidine-3,4,5-triol;
(2R,3S,4R,5R,6R)-N,N,N-Tris-N′,N′-bis-(2,3-dihydroxy-propyl)-5-(2,3-dihydroxy-propionylamino)-2,4,6-triiodo-isophthalamide-2,6-Bis-aminomethyl-piperidine-3,4,5-triol;
(2R,3S,4R,5R,6R)-N,N,N-Tris-N′-(2,3-dihydroxy-propyl)-5-(2,3,4-trihydroxy-butyrylamino)-2,4,6-triiodo-N′-methyl-isophthalamide-2,6-Bis-aminomethyl-piperidine-3,4,5-triol;
(2R,3S,4R,5R,6R)-N,N,N-Tris-N′-(2,3-dihydroxy-propyl)-5-(2,3,4-trihydroxy-butyrylamino)-2,4,6-triiodo-isophthalamide-2,6-Bis-aminomethyl-piperidine-3,4,5-triol;
(2R,3S,4R,5R,6R)-N,N,N-Tris-N′,N′-bis-(2,3-dihydroxy-propyl)-5-(2,3,4-trihydroxy-butyrylamino)-2,4,6-triiodo-isophthalamide-2,6-Bis-aminomethyl-piperidine-3,4,5-triol.

19. A diagnostic agent comprising a compound of formula (I) as defined in claim 1.

20. A diagnostic composition comprising a compound of formula (I) as defined in claim 1 together with pharmaceutically acceptable carriers or excipients.

21. An X-ray diagnostic composition comprising a compound of formula (I) as defined in claim 1 together with pharmaceutically acceptable carriers or excipients.

22. A method of diagnosis comprising administration of compounds of formula (I) as defined in claim 1 to the human or animal body, examining the body with a diagnostic device, compiling data from the examination and optionally analysing the data.

23. A method of imaging comprising administration of compounds of formula (I) as defined in claim 1 to the human or animal body, examining the body with a diagnostic device and compiling data from the examination and optionally analysing the data.

24. A method of X-ray imaging comprising administration of compounds of formula (I) as defined in claim 1 to the human or animal body, examining the body with a diagnostic device and compiling data from the examination and optionally analysing the data.
